(19) United States
(12) Patent Application Publication
(10) Pub. No.: US 2025/0006369 A1
(43) Pub. Date: Jan. 2, 2025

(54) SYSTEM AND METHOD OF ANALYSING DATA FOR THE DETERMINATION OF THE CONDITION OF A PERSON

(71) Applicant: Altoida, Inc., Washington, DC (US)

(72) Inventor: Ioannis Tarnanas, Washington, DC (US)

(21) Appl. No.: 18/708,503

(22) PCT Filed: Nov. 3, 2022
(86) PCT No.: PCT/IB2022/060595

§ 371 Date: May 8, 2024

Related U.S. Application Data
(60) Provisional application No. 63/277,456, filed on Nov. 9, 2021.

(51) Int. Cl. G16H 50/20; A61B 5/00

(52) U.S. Cl. CPC G16H 50/20; A61B 5/7282

(57) ABSTRACT

A system for determining the condition of a person comprises: obtaining a first set of digital biomarkers from a subject from sensors associated with the subject; generating from the first set of digital biomarkers a digital neuro signature; generating from the digital neuro signature a first digital neuro fingerprint, being a comparison of the biomarkers in the first digital neuro signature with threshold values associated with the first set of biomarkers; identifying from the first digital neuro fingerprint a first condition of the subject; obtaining a second set of biomarkers from one or more sensors and generating a second digital neuro signature and therefrom a second digital neuro fingerprint, being a comparison of the biomarkers in the second digital neuro signature with threshold values associated with the second set of biomarkers; identifying from the second digital neuro fingerprint a second condition of the subject; identifying in the first and second digital neuro fingerprints data patterns and utilising the patterns in the first and second digital fingerprints to identify the second condition in the first digital neuro fingerprint. The system and method can correlate biomarker data from different sources to determine the condition of a patient, including conditions not evident from that core data set. They can be used identifying the cognitive or physical condition of a person long before biological symptoms become evident by traditional diagnosis.

### FILED OF THE INVENTION

The present invention relates to a system and method of analysing data for the determination of the condition of a person. The preferred embodiments provide a system and method able to correlate data sets from different sources, particularly biomarker data, and to determine therefrom the condition of a patient, not only specific conditions related to a first, or core, data set but also other conditions not immediately evident from that core data set. The system and method can be used in the identification of the cognitive or physical condition of a person, often long before biological symptoms become evident by traditional diagnostic methods.

### BACKGROUND OF THE INVENTION

As is well documented in the art, the applicant has developed a system and method of analysing the condition of a person which generates a Digital Neuro Signature (DNS) based on a set of biomarkers obtained from one or more sensors configured to observe the person over the course of one or more activities. In the preferred practical implementations of the applicant's technology, the optimal DNS comprises 784 active digital biomarkers. The number of digital biomarkers used is not material to the teachings herein, which can be implemented with any chosen or desired set and number of biomarkers. A greater number of biomarkers can optimise the categorisation of a person's physical or cognitive condition, consistent with the system the applicant is currently implementing.

The applicant has developed a system for visualising a person's biomarkers as a matrix of which an example is shown in FIG. 1. This visualisation system is referred to hereinafter as a Digital Neuro Fingerprint (DNF).

The matrix of this example comprises a colour-based coding of the pixels forming the matrix. The colour code is used to represent the determined numerical value of the feature/biomarker. In the example shown, green is used to represent near the mean value for a feature/biomarker (based on an existing dataset for healthy controls), while red represents the limits of the curve in either direction (below or above the mean). Different shades of green and red correspond to the identified numerical values and how close they are to the mean.

The 784 active digital biomarkers of this example are produced by an Inertial Measurement Unit (IMU), of the type that can be found in mobile telephones and other wearable devices, such as smart watches and smart bracelets (an example being the FitBit™). The methodology for obtaining the active digital biomarkers is disclosed in detail in the applicant's earlier patent applications and more recently in many widely available publications in the art. The methodology is not determinative of the teachings herein.

The DNF provided by the matrix of FIG. 1 is a very useful tool in analysing the condition of a person. It can provide a visual matrix from which patterns can be readily identified and correlated between subjects, typically between subjects known to be healthy or known to have a particular illness or disease. The system and method can be used, for example, to identify cognitive impairment such as Alzheimer's, although they can be used for many other applications beyond cognitive function.

The system has been found to be able to identify the onset of cognitive impairment a significant time before traditional methods are able to do so. Early identification of diseases such as Alzheimer's can enable much more effective early treatment and significantly slow the development of the disease, if not permit a cure.

The technologies the applicant has developed, for diagnosing early onset of diseases such as Alzheimer's and other cognitive impairment, have been widely reported. Additionally, in the summer of 2021 the applicant was awarded FDA Breakthrough Designation for the development of world's first precision neurology device for the prediction of Alzheimer's disease.

The system developed by the applicant is able to capture multidimensional digital biomarkers and is not limited to latency- or accuracy-based measures. It is able to integrate several objectively measured features into a single task. This integration can increase the ecological validity of the observations, as it can create a more generalisable ‘real-world situation’ than in traditional laboratory test settings. The abundance of data collected by applicant's system both by the novel combination of multiple variables addressing across multiple cognitive domains as well as sensor data, yields a higher sensitivity, particularly when variability measures are considered. The digital biomarker platform produces significant volumes of high-resolution data that may include cognitive and motor processing; voice-based data that are indicative of the affective state and micro-errors that divulge where, when, and how a disease manifestation is affecting everyday function. These data have the potential to be further leveraged for disease progression modelling, for more accurate conversion event prediction or modelling of drug effects, leading to at-scale, non-intrusive lifelong monitoring of brain health.

The teachings herein build on the applicant's well documented existing technology.

### SUMMARY OF THE INVENTION

The present invention seeks to provide a system and method able to extend the technology described above and preferably in ways that enables the identification of other diseases and illnesses that are not currently identifiable. In the preferred embodiments this is achieved by collecting data from other sources, correlating this with an existing or core Digital Neuro Fingerprint to produce a new Digital Neuro Fingerprint, from which it is possible to identify data patterns and sets in the original Digital Neuro Fingerprint and associated Signature indicative of those other illnesses or diseases and not identifiable from the core fingerprint/signature alone. In another aspect, the data from other sources (typically other sensors) is used to enhance a Digital Neuro Fingerprint to provide better identification of a person's condition and/or to develop a new Digital Neuro Fingerprint that can identify a first condition based on the other sensor sources.

According to an aspect of the present invention, there is provided a method of determining the condition of a person, comprising:
obtaining a first set of digital biomarkers from one or more sensors associated with a subject;
generating from the first set of digital biomarkers a first digital neuro signature for that subject;
generating from the first digital neuro signature a first digital neuro fingerprint, the first digital neuro fingerprint being a comparison of the biomarkers in the first digital neuro signature with threshold values associated with the first set of biomarkers;
identifying from the first digital neuro fingerprint a first condition of the subject;
obtaining a second set of biomarkers from one or more sensors and generating therefrom a second digital neuro signature;
generating from the second digital neuro signature a second digital neuro fingerprint, the second digital neuro fingerprint being a comparison of the biomarkers in the second digital neuro signature with threshold values associated with the second set of biomarkers;
identifying from the second digital neuro fingerprint a second condition of the subject;
identifying in the first and second digital neuro fingerprints any data patterns; and
utilising any identified patterns in the first and second digital fingerprints to identify the second condition in the first digital neuro fingerprint.

Advantageously, the first and second sets of biomarkers are obtained for different subjects.

The first and second sets of biomarkers may be associated with different conditions, such as different stages of a disease or illness, for example on the one hand a preclinical stage of a disease or illness and on the other hand a prodromal or clinical stage of a disease or illness. The first and second sets of biomarkers may be associated with different diseases or illnesses.

In an embodiment, any identified patterns in the first and second digital fingerprints are used to enhance a determination of the first condition in the first digital neuro fingerprint.

Preferably, the threshold values are representative of an average value of that biomarker in a group of subjects considered to have a predetermined state, which may be a healthy state.

The method preferably includes the steps of:
generating from a set of said first digital neuro fingerprints obtained from a set of subjects a prototypical first digital neuro fingerprint,
generating from a set of said second digital neuro fingerprints a prototypical second digital neuro fingerprint,
wherein the step of identifying any data patterns is performed on the first and second prototypical digital neuro fingerprints.

Advantageously, the method includes the steps of:
generating a new first digital neuro fingerprint from any data pattern matches determined in the identification step, wherein the new first digital neuro fingerprint is indicative of the second condition in the subject.

In the preferred embodiments, the digital neuro fingerprints are formed of a plurality of elements, each associated with a biomarker, and in which each element has a value indicative of a deviation of the measured biomarker relative to the threshold. The values of each of the digital neuro fingerprints are advantageously in a given range. In the preferred embodiment, the value of each element is an optical value, the optical value preferably being a colour that changes in dependence on the deviation of a measured biomarker relative to the associated threshold. The step of identifying patterns in the first and second digital neuro fingerprints is preferably by optical pattern recognition.

It will be appreciated that colour pattern recognition is just one example implementation. Other systems could be used, including for example numerical or other indicia, greyscale indicators, black and white patterns and so on.

In one embodiment, the first and second sets of biomarkers are obtained from different sensors or sensor sets.

The method may include the step of generating a combined digital neuro fingerprint from the first and second digital neuro fingerprints, and utilising the combined digital neuro fingerprint in the determination of the first and/or second condition of the subject.

The method may include the step of generating a new digital neuro fingerprint from the first and second digital neuro fingerprints, and utilising the new digital neuro fingerprint in the determination of the first and/or second condition of the subject.

Advantageously, the first and/or second set of biomarkers are obtained from one or more of: a smartphone, a tablet computer, a smart watch, a smart bracelet, a pair of smart glasses, and a camera.

In the preferred embodiments, the method can be used in the diagnosis of diseases or disease categories; for example in the diagnosis of one or more of: Parkinson's, Alzheimer's, ALS.

Preferably, a match is identified if a number of elements in the first and second digital neuro fingerprints exceeds a set proportion. A match may for example be identified if a number of elements in the first and second digital neuro fingerprints exceeds 80% of the total number of elements in at least one of the digital neuro fingerprints. It is to be understood that the threshold value can be chosen by the skilled person and may vary from the example given. A lower threshold will find more pattern matches between data sets but will be potentially less reliable, whereas a higher threshold is likely to find fewer matches but will be more reliable.

Preferably, the step of identifying a pattern generates and uses Shapley Values.

According to another aspect of the present invention, there is provided a system for determining the condition of a person comprising:
one or more sensors associated with a subject, the or each sensor being operable to obtain one or more biomarkers of the subject;
a processing unit for processing the biomarkers, the processing unit comprising:
   a first register containing a first set of digital biomarkers from a subject obtained from one or more sensors associated with the subject;
   a first digital neuro signal generator configured to generate from the first set of digital biomarkers a first digital neuro signature for that subject;
   a first digital neuro fingerprint generator configured to generate from the first digital neuro signature a first digital neuro fingerprint, the first digital neuro fingerprint being a comparison of the biomarkers in the first digital neuro signature with threshold values associated with the first set of biomarkers;
   an identifying processor configured to identify from the first digital neuro fingerprint a first condition of the subject;
   an input configured to receive a second set of biomarkers from one or more sensors;
   a second digital neuro signature generator configured to generate from a second set of biomarkers a second digital neuro signature;
   a second digital neuro signature generator configured to generate from the second digital neuro signature a second digital neuro fingerprint, the second digital neuro fingerprint being a comparison of the biomarkers in the second digital neuro signature with threshold values associated with the second set of biomarkers;
   a data pattern identifier configured to identify in the first and second digital neuro fingerprints any data patterns;
the processing unit being configured to utilise any identified patterns in the first and second digital fingerprints to identify the second condition in the first digital neuro fingerprint.

The first and second sets of biomarkers may be obtained from different subjects. The first and second sets of biomarkers may be associated with different conditions. They may be associated with different stages of a disease or illness, for example the first set of biomarkers may be associated with a preclinical stage of a disease or illness and the second set of biomarkers may be associated with a prodromal or clinical stage of a disease or illness.

Preferably, the processing unit is configured to generate an enhanced determination of the first condition from any identified patterns in the first and second digital fingerprints.

Advantageously, the threshold values are representative of an average value of that biomarker in a group of subjects considered to have a predetermined state.

The system may include: a prototypical first digital neuro fingerprint generator configured to generate from a set of first digital neuro fingerprints obtained from a set of subjects a prototypical first digital neuro fingerprint, a prototypical second digital neuro fingerprint generator configured to generate from a set of second digital neuro fingerprints a prototypical second digital neuro fingerprint, wherein the processing unit is configured to identify any data patterns from the first and second prototypical digital neuro fingerprints.

The system may include: a new first digital neuro fingerprint generator configured to generate a new first digital neuro fingerprint from any data pattern matches determined in the identification step, wherein the new first digital neuro fingerprint is indicative of the second condition in the subject.

In preferred embodiments, the first and second digital neuro fingerprint generators are configured to form the first and second digital neuro fingerprints as an array of elements, each associated with a biomarker, and in which each element has a value indicative of a deviation of the measured biomarker relative to the threshold.

In a practical embodiment, the value of each element is an optical value, each element is a pixel of a display and the system comprises an optical sensor for determining the optical values. For this purpose, the processing unit may comprise an optical pattern identification unit.

The system may include a combined digital neuro fingerprint generator configured to generate a combined digital neuro fingerprint from the first and second digital neuro fingerprints, the processing unit being configured to utilise the combined digital neuro fingerprint in the determination of the first and/or second condition of the subject.

The system may include a new digital neuro fingerprint generator configured to generate a new digital neuro fingerprint from the first and second digital neuro fingerprints, the processing unit being configured to utilise the new digital neuro fingerprint in the determination of the first and/or second condition of the subject.

Advantageously, the system includes one or more of: a smartphone, a tablet computer, a smart watch, a smart bracelet, a pair of smart glasses, and a camera, configured to obtain the first and/or second set of biomarkers.

The system advantageously includes a diagnostic unit configured to diagnose a disease or disease categories; such as one or more of: Parkinson's, Alzheimer's, ALS.

According to another aspect of the present invention, there is provided a method of determining the condition of a person, comprising:
obtaining a first set of digital biomarkers from a first set of sensors associated with a subject;
generating from the first set of digital biomarkers a digital neuro signature for that subject;
generating from the digital neuro signature a first digital neuro fingerprint, the first digital neuro fingerprint being a comparison of the biomarkers in the first digital neuro signature with threshold values associated with the first set of biomarkers;
identifying from the first digital neuro fingerprint a first condition of the subject;
obtaining a second set of biomarkers from a second set of sensors different from the first set of sensors and generating therefrom a second digital neuro signature;
generating from the second digital neuro signature a second digital neuro fingerprint, the second digital neuro fingerprint being a comparison of the biomarkers in the second digital neuro signature with threshold values associated with the second set of biomarkers;
identifying in the first and second digital neuro fingerprints any data patterns; and
utilising any identified patterns in the first and second digital fingerprints to enhance the identification of the first condition of the subject.

The first and second sets of biomarkers may be obtained from different subjects.

Advantageously, the first and second sets of biomarkers are associated with different conditions, for instance with different stages of a disease or illness, and/or with different diseases or illnesses.

The threshold values may be representative of an average value of that biomarker in a group of subjects considered to have a predetermined state, of instance a healthy state.

The method may include the steps of: generating from a set of first digital neuro fingerprints obtained from a set of subjects a prototypical first digital neuro fingerprint, generating from a set of second digital neuro fingerprints a prototypical second digital neuro fingerprint, wherein the step of identifying any data patterns is performed on the first and second prototypical digital neuro fingerprints.

The method may include the steps of: generating a new first digital neuro fingerprint from any data pattern matches determined in the identification step, wherein the new first digital neuro fingerprint is indicative of the first condition of the subject.

In a practical embodiment, the digital neuro fingerprints are formed of a plurality of elements, each associated with a biomarker, and in which each element has a value indicative of a deviation of the measured biomarker relative to the threshold.

The method preferably includes the step of identifying patterns in the first and second digital neuro fingerprints by optical pattern recognition.

The method may include the step of generating a combined digital neuro fingerprint from the first and second digital neuro fingerprints, and utilising the combined digital neuro fingerprint in the determination of the first and/or a second condition of the subject.

The method may include the step of generating a new digital neuro fingerprint from the first and second digital neuro fingerprints, and utilising the new digital neuro fingerprint in the determination of the first and/or a second condition of the subject.

According to another aspect of the present invention, there is provided a system for determining the condition of a person comprising:
a first set of sensors associated with a subject, the or each sensor being operable to obtain one or more biomarkers of the subject;
a processing unit for processing the biomarkers, the processing unit comprising:
   a first register containing a first set of digital biomarkers from a subject obtained from the first set of sensors associated with the subject,
   a first digital neuro signal generator configured to generate from the first set of digital biomarkers a first digital neuro signature for that subject;
   a first digital neuro fingerprint generator configured to generate from the first digital neuro signature a first digital neuro fingerprint, the first digital neuro fingerprint being a comparison of the biomarkers in the first digital neuro signature with threshold values associated with the first set of biomarkers;
   an identifying processor configured to identify from the first digital neuro fingerprint a first condition of the subject;
   an input configured to receive a second set of biomarkers from a second set of sensors different from the first set of sensors and generating therefrom a second digital neuro signature;
   a second digital neuro signature generator configured to generate from the second set of biomarkers a second digital neuro signature;
   a second digital neuro signature generator configured to generate from the second digital neuro signature a second digital neuro fingerprint, the second digital neuro fingerprint being a comparison of the biomarkers in the second digital neuro signature with threshold values associated with the second set of biomarkers;
   a data pattern identifier configured to identify in the first and second digital neuro fingerprints any data patterns;
the processing unit being configured to utilise any identified patterns in the first and second digital fingerprints to enhance the identification of the first condition of the subject.

While different aspects of the invention disclosed herein are set out above and in the claims, it is to be understood that these aspects are combinable together into a single method and system and also that their elements are equally combinable with one another.

The preferred embodiments of the present invention provide a system and method of creating novel Digital Neuro Fingerprints (DNFs) using data sets from a base Digital Neuro Fingerprint (DNF) enhanced with data obtained from other sources. The additional data may be collected from other devices containing an Inertial Measurement Unit, although it could be collected from any other data source using other sensors, as explained below.

The system and method are configured to correlate data from such other sources into a common characterisation framework, in this example the colour coding scheme shown in FIG. 1, and most preferably common with the framework used in the base DNF generated from the base Digital Neuro Signature. The additional data can be used to generate a combined Digital Neuro Fingerprint or new combinations of Digital Neuro Fingerprints, which can be used in verifying not only earlier known conditions but also in identifying potential new conditions of a patient not immediately derivable from the core DNF.

In the preferred embodiments, the system and method make use of optical pattern recognition to locate data patterns useful in the identification of physical or cognitive conditions.

In a practical embodiment, the system and method provide for:
(a) the creation of new DNFs inside datasets that didn't originally collect data using a device for the collection of the base set of biomarkers, using other devices containing an IMU or other sensor. Examples include a smart watch or bracelet such as a FitBit™, Apple Watch™ and so on;
(b) the mathematical processing of these DNFs using an optical pattern recognition apparatus or method;
(c) the creation of combined DNFs or DNF combinations, using the core set of active digital biomarkers and/or novel digital biomarkers that utilize sensors other than an IMU.

A primary advantage of the system and method disclosed herein is to allow rapid re-purposing of a DNF obtained from one source, such as the devices the subject of the applicant's earlier filed patent applications, to new disease areas, on the basis of DNF similarities. Once such similarities are found in a combined DNF or a new combination of DNFs, a clinical trial for the validation of the identification of a new disease area will require significantly fewer patients and could have a reduced study duration, thus making the clinical trial more efficient and agile.

According to another aspect of the present invention, there is provided the use of a determination of a subject's identified condition obtained by a method or by a system as claimed in any preceding claim to provide a pharmaceutical or other intervention to treat the identified condition.

According to another aspect of the present invention, there is provided a method of treating a subject on the basis of a determination of a subject's identified condition obtained by a method or by a system as claimed in any preceding claim to provide a pharmaceutical or other intervention to treat the identified condition.

In the use or method, the suggested intervention may be a pharmaceutical intervention and the information relates to the identity of a specific drug to be administered to the individual.

In the use or method, the drug may be a cholinesterase inhibitor (such as donepezil, rivastigmine or galantamine), memantine (optionally in combination with a cholinesterase inhibitor), a monoclonal antibody (such as Aducanumab (Aduhelm), BAN2401, gantenerumab (optionally in combination with solanezumab), solanezumab (optionally in combination with gantenerumab), a sigma-1 receptor agonist (optionally also M2 autoreceptor antagonist or NMDA receptor antagonist, such as ANAVEX2 (blarcamesine), AVP-786 or AXS-05), an SV2A modulator (such as AGB101 (low-dose levetiracetam), a mast-cell stabiliser (such as ALZT-OP1 (cromolyn+ibuprofen)), an anti-inflammatory (such as ALZT-OP1 (cromolyn+ibuprofen)), a RAGE antagonist (such as azeliragon), a glutamate modulator (such as BHV4157 (troriluzole)), a D2 receptor partial agonist (such as brexpiprazole), serotonin-dopamine modulator (such as brexpiprazole), an amyloid vaccine (such as CAD106), a bacterial protease inhibitor (such as COR388), a selective serotonin reuptake inhibitor (such as escitalopram), an antioxidant (such as Ginkgo biloba), a plant extract (such as Ginkgo biloba), an alpha-2 adrenergic agonist (such as guanfacine), an omega-3 fatty acid (such as icosapent ethyl (IPE), which is a purified form of eicosapentaenoic acid), an angiotensin II receptor blocker (such as losartan), a calcium channel blocker (such as amiodipine), a cholesterol agent (such as atorvastatin), a combination of an angiotensin II receptor blocker (such as losartan), a calcium channel blocker (such as amiodipine), a cholesterol agent (such as atorvastatin) with or without exercise, a tyrosine kinase inhibitor (such as masitinib), an insulin sensitiser (such as metformin), a dopamine reuptake inhibitor (such as methylphenidate), an alpha-1 antagonist (such as mirtazapine), an acetylcholinesterase inhibitor (such as octohydro-aminoacridine succinate), a ketone body stimulant (such as tricaprilin), a caprylic triglyceride (such as tricaprilin), a Tau protein aggregation inhibitor (such as AADvac1 or TRx0237 (LMTX)), a positive allosteric modulator of GABA-A receptors (Zolpidem and zoplicone), or BPDO-1603, or combinations of any of these to be administered either together or separately.

In the use or method, Aducanumab (Aduhelm) is preferably administered to the subject.

In the use or method, the suggested intervention may be a pharmaceutical intervention and the information relates to the frequency and/or dose of the pharmaceutical intervention or specific drug to be administered to the individual.

In the use or method, the individual may have been previously diagnosed with mild cognitive impairment and the information relating to a pharmaceutical or other intervention provided by the information output relates to whether a previously prescribed intervention is effective in that individual.

Other advantages and aspects of the present invention will be apparent to the skilled person from the following detailed description.

### BRIEF DESCRIPTION OF THE DRAWINGS

Embodiments of the present invention are described below, by way of example only, with reference to the accompanying drawings, in which:

FIG. 1 is an example of a Digital Neuro Fingerprint generated from a digital Neuro Signature itself generated from a set of biomarkers obtained from a person;

FIG. 2 shows a flow chart of one embodiment of system and method for determining patterns matches in Digital Neuro fingerprints from a plurality of different subject sets;

FIG. 3 shows an example of pattern matching;

FIG. 4 shows an example of pattern matching using Shapley values;

FIG. 5 is an example of a pattern match matrix;

FIG. 6 is an example of a combined Digital Neuro Fingerprint;

FIG. 7 is a flow chart of an embodiment of system and method for producing new and combined Digital Neuro Fingerprints according to the teachings herein;

FIG. 8 is a schematic diagram of an embodiment of system according to the teachings herein; and

FIG. 9 is a schematic diagram of an embodiment of processing unit according to the teachings herein.

### DESCRIPTION OF THE PREFERRED EMBODIMENTS

### Active Biomarkers

Biomarkers are the heart of disease diagnostics. A biomarker, or biological marker, refers to a parameter that can be measured to indicate reliably and accurately the presence and severity of a condition. Biomarkers may include a wide array of measurable indicators ranging anywhere from an elevated white blood cell count to indicate infection, to the presence of beta-amyloid plaques in the brain to indicate Alzheimer's disease.

With the rise of digital health data collection, researchers and providers alike are embracing the potential of digital biomarkers. The applicant's system collects clinically meaningful data through digital devices, which the applicant has found can provide a new and more robust method for monitoring and diagnosing the condition of a person. They allow for the collection and analysis of physiological and behavioural data, which can be used for predictive diagnosis of diseases.

Digital biomarkers can provide and facilitate early diagnosis to gain earlier access to treatments and therapies when the disease is more treatable, providing better health outcomes.

Digital biomarkers are quantifiable, typically objective physiological and behavioural data that can be collected and measured via a digital device. Example devices include portables, wearables, implantables, or digestibles. Digital biomarkers yield robust data sets that can be used to learn more about the nuances of specific diseases and gain valuable health insight.

Passive data from sensors integrated into wearable devices, such as smart watches, is generated when a user simply wears the device. The data collected is then referred to as passive digital biomarker data. Similarly, digital biomarker data can be generated and captured from smart devices, such as smartphones and tablets, when a user interacts with the device in response to an active prompt. Integrated or separate sensors including cameras, microphones, touchscreen sensors, accelerometers, and gyroscopes can be used to collect active digital biomarker data.

While wearables collect more obvious data, such as: heart rate, heart rate variability, and oxygen saturation levels from photoplethysmography sensors; smartphones and tablets collect less intuitive, yet incredibly powerful data. Some examples of digital biomarker data that can be collected from smart devices include:
(a) microphones, which can be used to detect biomarkers of speech, such as fluency, mood, and sentiment;
(b) cameras, which can detect eye movements, pupil dilation, and facial expression;
(c) touchscreen sensors, which can identify fine motor skills required for tapping, swiping, and typing;
(d) inertial sensors, including accelerometers and gyroscopes, which can detect human motion and posture, enabling the measurement of gait metrics.

The number and types of biomarkers used can provide, the applicant has found, a comprehensive view of a person's condition. They can comprise motoric markers, including for example speed of movement, range of movement, force applied in an action (pressing a button or pressure sensitive pad for example) trembling, reaction times, and so on; and they may also comprise cognitive indicators, such as time to complete a task, accuracy of movement or reaction, accuracy and time to complete a set exercise and so on.

Digital biomarkers can also provide longitudinal (time lapse) data collection both at an individual and at a population level. Most tools used to assess brain health lack the infrastructure for longitudinal analysis and typically only provide a means of cross-sectional data collection and analysis. Longitudinal data offers the ability to analyse brain health on a personalized basis to gain insights into how an individual's brain health is changing over time. With longitudinal data, which tracks the progression of digital biomarkers over time, a processing unit (preferably an artificial intelligence) can make predictions from data to determine if, when, and how an individual will develop a particular illness or disease such as Alzheimer's.

As more and more individuals embrace newer health-related technologies, the amount of available health data is growing at a staggering rate. When this volume of data is paired with strong analytical tools, it can potentially be leveraged to track trends and patterns for many diseases.

The applicant has developed a precision neurology platform that can measure and analyse a wide range of cognitive and functional digital biomarkers, providing a comprehensive analysis of neurocognitive and more general function on a personalized level. The platform has been developed to collect and analyse in the preferred implementation nearly 800 active digital biomarkers, which enable highly specific, accurate, and generalizable data for both cross-sectional and longitudinal analysis of a person's health.

Further information relating to the applicant's system and method for generating such a dataset and Digital Neuro Signatures (DNS) is disclosed in U.S. Patent Application No. 63/211,953, filed on 17 Jun. 2021, which is incorporated herein in its entirety by reference.

### Creation of Core DNFs for a Subject

The system and method are configured to generate a dataset from sensors that are generally widely available. This may come, for example, from wearable sensors. These may, for example, comprise one or more of the following: PIR motion sensors, body-worn sensors (usually containing an IMU), pressure sensors, video monitoring, sound recognition and so on. The primary embodiments use in preference: (i) body-worn sensors and (ii) video monitoring. The signals from the sensors are compared with a predetermined set of biomarkers and from this comparison a core Digital Neuro Fingerprint is developed, based on whether the output of the sensor(s) meets a predetermined condition related to that biomarker. When a plurality of sensors is used, the preferred embodiments treat that condition as having been met if the output of just one of the sensors indicates this. In other embodiments, the system and method may be configured to require the output of a plurality of sensors or for each sensor to meet the condition, or for some other correlation in outputs to be met.

The predetermined condition may be the response that would be obtained from a healthy individual, although in other embodiments this may be the response obtained from an individual having an identified condition (such as a cognitive impairment, just as one example). Any reliable response, whatever that may be, could be used as the standard.

### Use Cases/Embodiments

In one embodiment according to the teachings herein, there can be provided a public domain dataset for a particular disease or illness (Parkinson's for example) that contains both body-worn sensor data points (e.g. a smart watch or bracelet such as a FitBit™) and a video monitoring modality (e.g. a home camera). In this embodiment, a researcher in the field can segment the data stream from the body-worn sensor into IMU outputs that correspond to specific activity associated with daily living “actions” captured by the video monitoring system. Those actions will most typically have a set duration, for instance 30-90 seconds or more. One practical example is an IMU sensor output obtained while a person is walking to find their car keys or while walking in the living room searching for an object such as a remote control of a television. An example of such datasets is the CART home by ORCATECH and many others: https://www.ohsu.edu/collaborative-aging-research-using-technology/cart-home.

Once the IMU outputs are segmented, they can be mapped to the set of digital biomarkers of a Digital Neuro Signature (such as the Altoida 784 DNS) by someone with knowledge of that Digital Neuro Signature. The result of this process is the creation of a set (in this example a set of 784) different numerical inputs that correspond to the Digital Neuro Signature and have a duration similar to the “action” time windows of the associated action, such as time walking to find keys, time to find a remote control, and so on. The sum of those time windows will typically be anything from 2 to 10 minutes.

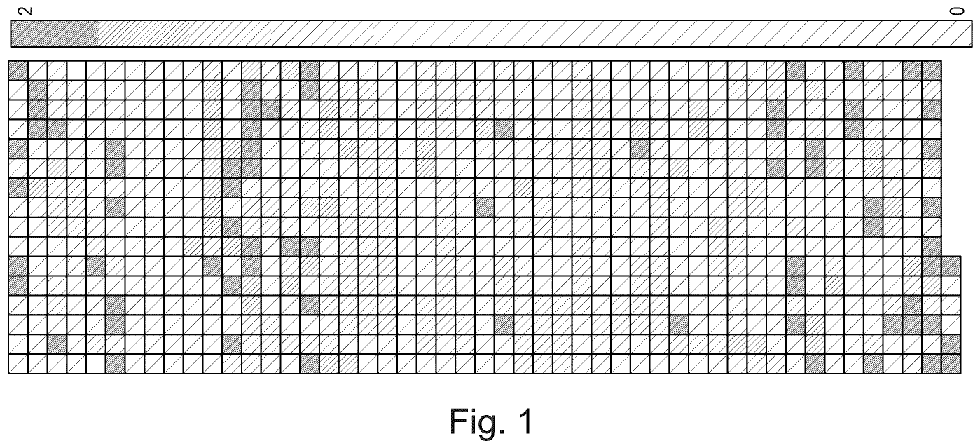

The Digital Neuro Signature is then mapped to a reference matrix to produce a Digital Neuro Fingerprint at the end of each session, of which the matrix of FIG. 1 is an example. In practical terms, it could be expected that such processing would take in the region of 10 minutes. The process can be automated once set up, so as to achieve the mapping in a rapid time.

The different numerical inputs are preferably associated with particular movements or other measurable characteristics.

In another embodiment, a public domain, that is the available or used sensors, comprises only body-worn sensor data points and no video monitoring modality. This embodiment of anecdotal information, for instance from the subject's actions during the previous day, is used to segment the IMU output into time frames that correspond to the activity of the daily living “actions”. Once those actions have been completed, the same process as above is followed. In this particular example, the resulting Digital Neuro Fingerprint might contain more noise than the dataset that has both body-worn and video monitoring data points. However, that noise can be accounted for in the subsequent processing or by relying on a larger data set.

### Neural Network Processing of New DNFs

The preferred methodology in this example produces a “prototypical DNF” for various disease categories in various disease datasets, e.g. Parkinson's, Alzheimer's, ALS etc, and compares them together, using an optical matching algorithm.

### Use Cases/Embodiments

In one embodiment, a public domain dataset for Parkinson's disease fulfils the conditions mentioned above for the creation of one or more Digital Neuro Fingerprints and in this example contains the following categories of patients:
a) Preclinical PD (n=100),
b) Prodromal PD (n=50),
c) clinical PD (n=500), etc.

The first step in the process is to create the Digital Neuro Fingerprints for each of these disease categories, that is:
100 Digital Neuro Fingerprints for Preclinical subjects
50 Digital Neuro Fingerprints for Prodromal subjects, and
500 Digital Neuro Fingerprints for clinical PD subjects.

Subsequently, the following algorithm is applied, in order to create one “prototypical” Digital Neuro Fingerprint for each of these categories: one for Preclinical, one for Prodromal and one for clinical PD.

For each of the Digital Neuro Fingerprint squares at each category, e.g. Preclinical subjects, the algorithm starts at the top left square and counts how many out of the 100 cases for Preclinical subjects were: red, green, light green, light red and so on. The majority wins. For example, if inside the 100 cases for Preclinical PD subjects there were: 70 red, 20 light red, 7 light green and 3 green biomarkers, the “prototypical” Digital Neuro Fingerprint for the Preclinical PD subject category will have the top left square (1st one in this process) set as red. The same goes for the remaining 783 squares, until a new “prototypical Digital Neuro Fingerprint” is produced for the category of Preclinical PD subjects.

The same process is preformed for each of the other categories in this dataset, thereby generating three “prototypical” Digital Neuro Fingerprints. For this particular algorithm, the numerical value that produces the colour coding is not material. The process simply ranks the colour coding for the production of the “prototypical” Digital Neuro Fingerprints. The end result might be a slightly less precise representation of the mean digital biomarkers value for this category, but this is acceptable for this embodiment.

The same process is repeated for the second dataset for this comparison, which for example might be an Alzheimer's one from the applicant's existing database, and having the following categories:
a) Preclinical AD (n=20),
b) Prodromal AD (n=500), and
c) clinical PD (n=50), etc.

A total of 3 prototypical Digital Neuro Fingerprints will be produced for the Alzheimer's dataset using the same algorithm as above.

The final step in the process is the optical pattern recognition on the basis of the prototypical Digital Neuro Fingerprints. An artificial neural network or another pattern recognition algorithm is employed to compare the six prototypical Digital Neuro Fingerprints generated in the above example for similarities. An acceptable match in this embodiment is >80%. Other embodiments may set the match threshold at a higher or lower percentage as desired.

Once such a match is found, it might for example be revealed that the prototypical Digital Neuro Fingerprint for Preclinical PD subjects has an >80% match with the prototypical Digital Neuro Fingerprint for Prodromal AD subjects and >90% match with the prototypical Digital Neuro Fingerprint for Preclinical AD subjects, and so on.

The Digital Neuro Fingerprints that match the most are then selected and reverse engineered to create a clinical profile based on the clinical information that exists in the datasets. For example, the prototypical Digital Neuro Fingerprint for Preclinical PD subjects, based on the colour coding, may be most frequently associated with sleep disorders in 59% of subjects, depressive syndrome in 76% of subjects and visual hallucinations in 74% of subjects. In addition, the prototypical Preclinical AD Digital Neuro Fingerprints that matches this one before from the Preclinical PD dataset might be most frequently associated with micromovement disorders in 61% of subjects, mood disorder in 46% of subjects and perceptual motor dysfunction in 64% of subjects.

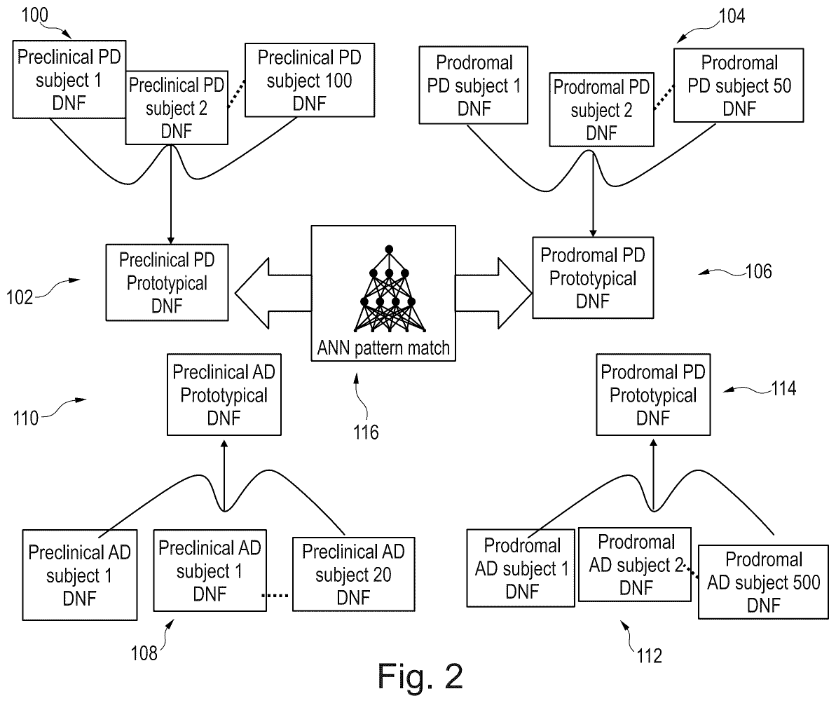

FIG. 2 shows an example flow chart implementing this process.

With reference to the flow chart, at step 100 the Digital Neuro Fingerprint of pre-clinical PD subjects 1-100 is collected and therefrom, at step 102 a prototypical Digital Neuro Fingerprint for Preclinical PD is produced. Similarly, at step 104 the Digital Neuro Fingerprints of Prodromal PD subjects is obtained and processed, at step 106, to generate a prototypical Digital Neuro Fingerprint for Prodromal PD. These are combined, as explained above, with the Digital Neuro Fingerprints for Alzheimer's disease in the following steps. At step 108 the Digital Neuro Fingerprints of Preclinical AD subjects (1-20 in this example) are obtained and therefrom, at step 110, a prototypical Digital Neuro Fingerprint for Preclinical AD is generated. Similarly, at step 112, the Digital Neuro Fingerprints for Prodromal AD subjects (in this example 500) are obtained and therefrom a prototypical Digital Neuro Fingerprint for Prodromal AD is generated at step 114.

At step 116, by means of a processing unit, preferably an artificial Neuro Network, patterns within the prototypical Digital Neuro Fingerprints are identified and from these patents new Digital Neuro Fingerprints can be developed for, in this example, Parkinson's disease and Alzheimer's disease, that have not previously been identified.

All of these reveal information that would help select the proper neuropsychological battery for a validation clinical trial, which would otherwise need to have a much more comprehensive neuropsychological battery for the data collection. It can also accelerate the conclusion of such a clinical trial for the validation, since the investigators are able to collect the newly created Digital Neuro Fingerprints from the clinical trial and see how well they match the “prototypical Digital Neuro Fingerprint” for the population using the same optical recognition algorithm above.

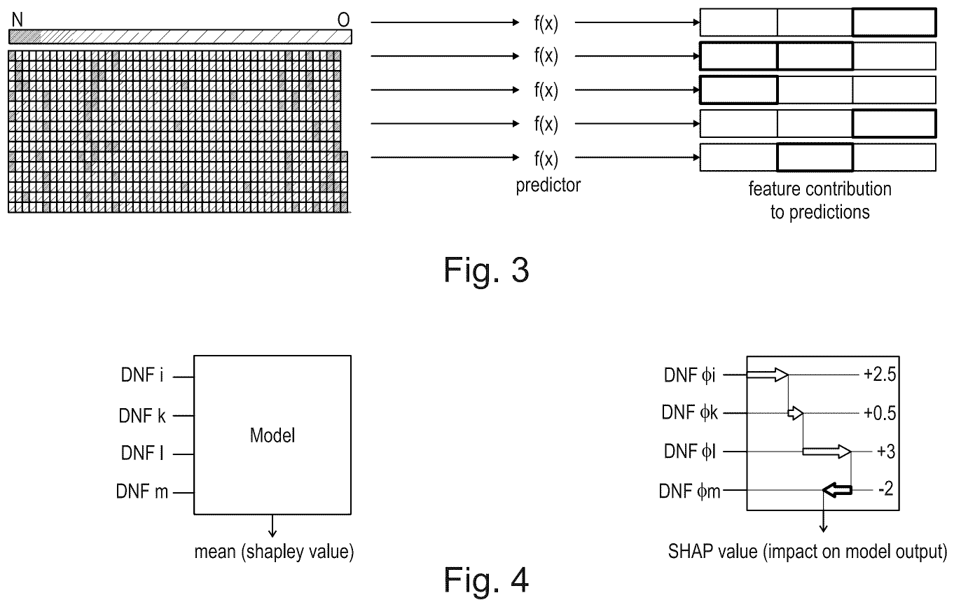

The methodology for reverse engineering to clinical profiles from the Prototypical Digital Neuro Fingerprint utilizes feature contribution to predictions using Shapley Values (see, for example: https://github.com/slundberg/shap) as Feature Interaction Scores (FIS) and estimates the value of data points based on Shapley impact on model output. Referring to FIGS. 3 and 4, once the square patterns corresponding to the FIS have been identified, the process creates a list of the most important coloured Digital Neuro Fingerprint squares i(1-784) and obtains their actual mean values, giving them a value ϕᵢ. The most important squares are those that are found most contribute to a match and that are most relevant to the associated biomarker. For example, for a biomarker related to analysis of a subject while performing an activity, the most important squares would be those more relevant to that activity, for example eye tracking, movement of the subject (such as, which way they turn). Other biomarkers, such as blood pressure or sleep pattern for example, would be deemed less relevant.

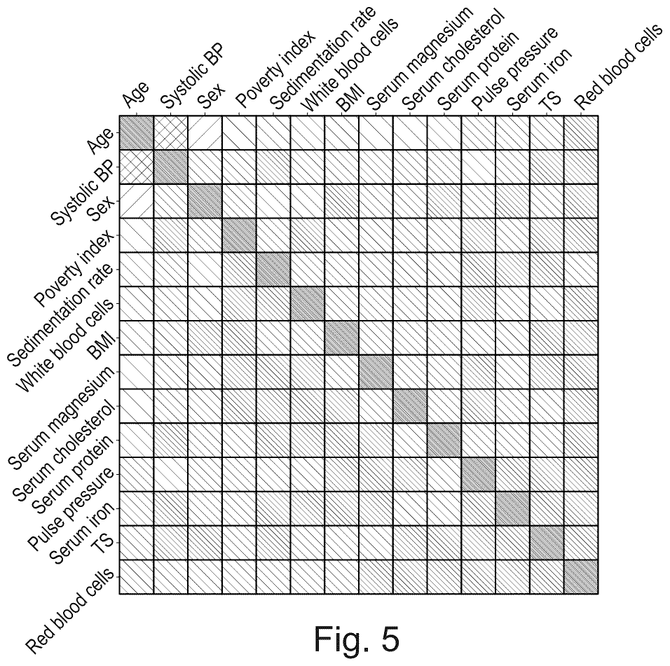

From this, the process the system calculates non-linear patterns between Digital Neuro Fingerprint ϕ_{i-m(or more)} above and the clinical data from the original dataset using XGBoost and SHAP “interaction effects”. The end result produces a matrix of the most relevant clinical features, which can reveal interesting interactions with Digital Neuro Fingerprint ϕ_{i-m(or more)}. An example is shown in FIG. 5.

### Creation of Combined DNFs or New DNF Combinations

In some cases a dataset contains novel data from IoT (Internet of Things) wearable sensors, e.g. grip strength data, which are not part of the set of (e.g. 784) DNS digital biomarkers. Similarly, the DNS digital biomarkers library could be updated with novel sensor data that are not part of an IMU. In these cases, the following procedure can be followed.

### (i) Use Cases/Embodiments

When a public domain dataset for Parkinson's disease, for example, contains novel data from a medical device, such as grip strength data and also body-worn sensor data points (for example from a FitBit™) or one video monitoring modality (for example from smart home cameras), the skilled person can segment the novel data stream from grip strength against either the body-worn IMU outputs or the video system outputs, as the first step of creating the Digital Neuro Fingerprints. Similarly, when no video system is used for the annotation, an anecdotal annotation can be used. The novel sensor numerical values are then be utilised to create a new independent Digital Neuro Fingerprint (separate to the core Digital Neuro Fingerprint that is created by the set of (e.g. 784) Digital Neuro Signature digital biomarkers).

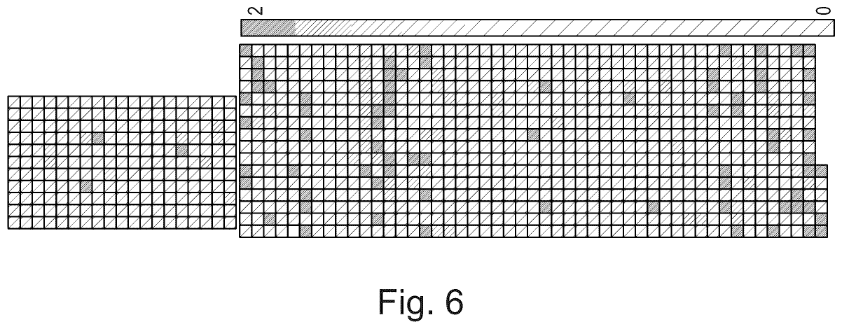

By way of explanatory example, the new Digital Neuro Fingerprint might have 120 Digital Neuro Fingerprint digital biomarkers, and is generated on the basis of the same colour coding rules, in other words the new Digital Neuro Fingerprint is generated using the identifier framework or system to allow for comparison with other Digital Neuro Fingerprints, whether generated from the same sensor devices or from other types of sensors. A possible visualization of the resulting Digital Neuro Fingerprint containing both the “core Digital Neuro Fingerprint” (e.g. 784 digital biomarkers) and the “novel Digital Neuro Fingerprint” (120 digital biomarkers) is shown in FIG. 6.

Essentially, a new combined Digital Neuro Fingerprint is created that combines the “core Digital Neuro Fingerprint” and the “novel Digital Neuro Fingerprint”. The shape of this combination could be made to start at the bottom left and add new shapes to it, for example “novel DNF 2”, “novel DNF 3” etc.

Following the creation of the combined Digital Neuro Fingerprint, a new “prototypical combined DNF” can be created for each of the disease categories of patients at the said dataset, for example: a) Preclinical PD (n=100), b) Prodromal PD (n=50), c) clinical PD (n=500), etc. The final step in this process is the optical pattern recognition on the basis of the “prototypical combined DNFs”.

When comparing a “prototypical combined DNFs” the following scenarios might happen: a) the prototypical combined Digital Neuro Fingerprints matching at >80%; b) matching only the core Digital Neuro Fingerprints at >80% and c) matching only the “novel” Digital Neuro Fingerprints at >80%. In the case of a prototypical combined Digital Neuro Fingerprint match scenario as above (a), the match is reverse engineered so that a clinical profile is created based on the clinical information that exists in the datasets. In the case of only the “core DNF” or “novel DNF” matching, the following procedure can be followed: the optical recognition search is repeated focusing on the unmatched Digital Neuro Fingerprint, for instance core or novel until a >80% match is found. Then those two independent matches, one for the “core DNF” and one for the “novel DNF” are used to create a new “prototypical combined DNF”, which is the outcome of a deeper search and not the result of original annotation based creation described above.

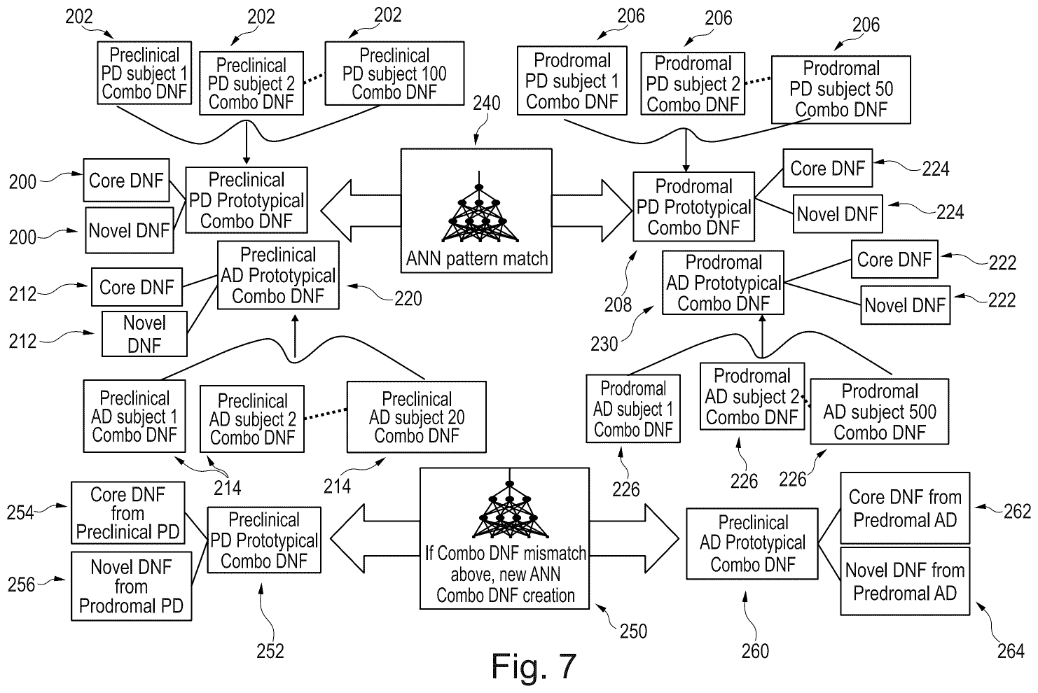

FIG. 7 shows an example flow chart implementing this process.

Referring to FIG. 7, in this example, at steps 200 core and novel Digital Neuro Fingerprints for Preclinical Parkinson's Disease are combined with Combined Digital Neuro Fingerprints from a plurality of subjects 1-100 (step 202) to generate a Prototypical Combined Digital Neuro Fingerprint for Preclinical Parkinson's disease. Alongside this, at step 204, core and novel Digital Neuro Fingerprints for Prodromal Parkinson's disease are combined with Combined Digital Neuro Fingerprints for a plurality of Prodromal PD subjects 1-50 (step 206) to generate a Prototypical Combined Digital Neuro Fingerprint for Prodromal Parkinson's disease (step 208).

Additionally, at step 212, core and novel Digital Neuro Fingerprints for Preclinical Alzheimer's disease are combined with Combined Digital Neuro Fingerprints from a plurality of Preclinical Alzheimer's Disease subjects 1-20 (step 214) to generate a Prototypical Combined Digital Neuro Fingerprint for Preclinical Alzheimer's disease, at step 220.

Furthermore, at step 222 core and novel Digital Neuro Fingerprints for Prodromal Alzheimer's disease are combined with Combined Digital Neuro Fingerprints for a plurality of Prodromal Alzheimer's disease subjects 1-50 (step 226) to generate (at step 230) a prototypical combined Digital Neuro Fingerprint for Prodromal Alzheimer's disease. These are pattern matched, at step 240, in a processing unit, preferably an artificial intelligence, as described above, to generate a new Prototypical Combined Digital Neuro Fingerprint, which is the outcome of a deeper search.

With reference to step 250 if the combined Digital Neuro Fingerprint is deemed to be a mismatch at step 240 a new combined Digital Neuro Fingerprint is created by the processing unit (or artificial neural network) on the basis of a Prototypical Combined Digital Neuro Fingerprint for Preclinical Parkinson's disease, at step 252, generated from a core Digital Neuro Fingerprint for Preclinical Parkinson's Disease subjects and a novel Digital Neuro Fingerprint for Prodromal Parkinson's disease subjects (steps 254 and 256 respectively). This is compared to a Prototypical Combined Digital Neuro Fingerprint for Preclinical Alzheimer's Disease generated at step 260 from a core Digital Neuro Fingerprint from Prodromal Alzheimer's Disease subjects and a novel Digital Neuro Fingerprint from Preclinical Alzheimer's Disease subjects (steps 262 and 264 respectively).

In another example, one of the datasets does not have any “novel DNFs” to be matched. In such a case, the match is performed using the “core DNFs” only and the match is reverse engineered so that a clinical profile is enriched by the novel data of the medical device, for instance grip strength. This creates a new clinical profile that might have unique clinical characteristics, which inform novel subtypes of a known disease, for example. While the earlier prototypical Digital Neuro Fingerprint case for Preclinical PD of this example was most frequently associated with sleep disorders in 59% of patients, depressive syndrome in 76% of patients and visual hallucinations in 74% of patients, the novel Digital Neuro Fingerprint might also reveal the reduction of grip strength at 34% of the patients.

In the case of “prototypical combined DNFs”, it could be that the novel Digital Neuro Fingerprint for the reduction of grip strength in PD matches a similar novel Digital Neuro Fingerprint for the reduction of grip strength in 25% of Prodromal AD patients. This is a novel subtype for Prodromal PD that can be validated by a clinical trial. All of the above examples show that the system and process are able to reveal information that would help select the proper neuropsychological battery for a validation clinical trial, which would otherwise need to have a much more comprehensive neuropsychological battery for the data collection. It can also accelerate the conclusion of the clinical trial for the validation, since the investigators are able to collect the newly created Digital Neuro Fingerprints from the clinical trial and see how well they match the “prototypical combined DNF” for that population using the same optical recognition algorithm described herein.

### Example Apparatus/System

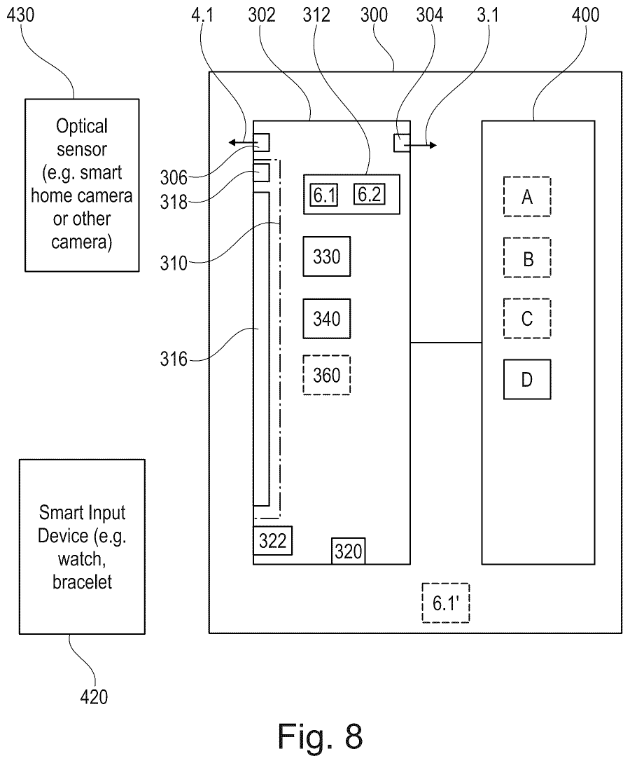

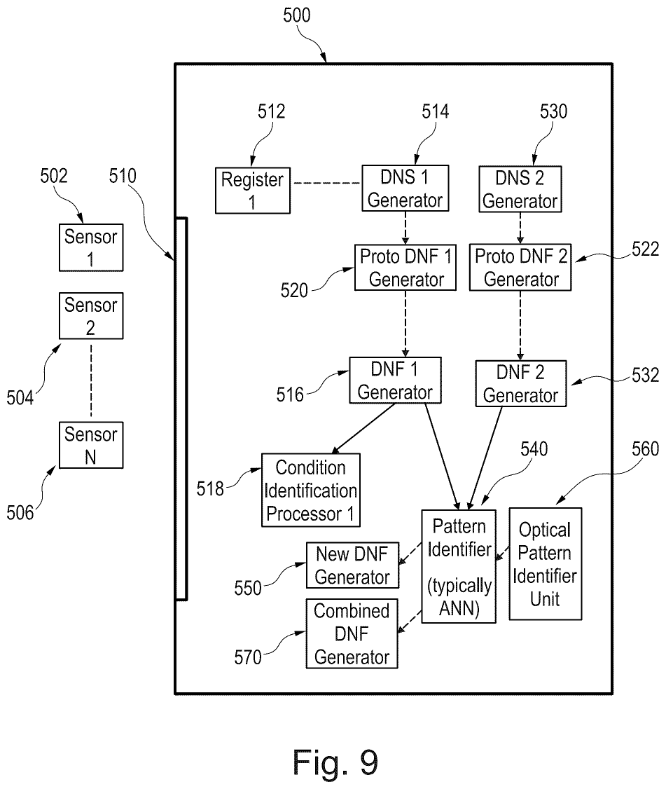

Examples of suitable apparatus for implementing the taught system and method will be immediately apparent to the skilled person from the above disclosure. Nevertheless, for the sake of completeness, an example is described below, with reference to FIG. 8.

FIG. 8 shows an embodiment of apparatus 300 for use in implementing the teachings herein. The apparatus 300 in this example comprises a mobile device 302, provided with first and second cameras 304, 306, typically one being front facing (away from the user) and the other being rear facing (towards the user and the same side as the display). The mobile device 300 typically also includes an output unit 310, a position sensor 312 (such as a GPS module, an accelerometer and so on), a microphone 320, a user input unit 322 and one or more processing units 330, 340, 360.

The mobile device 300 is preferably a handheld portable device like a smartphone. However, the mobile device 302 may also be any other user portable device. It may, for example, be wearable, such as a smart watch or bracelet, smart glasses or similar. The mobile device 300 may be a single device or implemented in a plurality of devices, such as a smart telephone in conjunction with a smart watch or bracelet, or even glasses. FIG. 8 shows such smart devices 420 as external accessories configured to communicate with the mobile device 300.

The output unit 310 may include a display 316 and in some implementations a projector, such as an eye projector in a pair of smart glasses. The output may also include an acoustic unit 318 such as a loudspeaker and/or audio output port for earphone or headphones.

There may be provided an internal device 400, typically a processing unit, advantageously an artificial neural network, for carrying out computational work remote from the mobile device 300, including but not limited to computation of data from a plurality of different subjects, as provided for in the above teachings. The processing unit 400 would typically be coupled to the mobile device 300 to exchange data, remotely such as through the internet, a wireless network or via the GSM network. In some implementations the processing until 400 may comprise a central processing computer. It is to be appreciated that in some embodiments all processing is carried out within the device 300.

The apparatus may also include, as described above, an external optical sensor such as a smart home camera or other camera 430 configured to obtain images of the subject and relaying them either to the mobile unit 300 or to the external processing unit 400 or to both. It will be appreciated that the external optical unit 430 may comprise a set of cameras or the like, able to obtain a plurality of images of a subject, whether sequentially or simultaneously.

An example of a processing unit according to the teachings herein is shown in FIG. 8.

The system comprises a plurality of sensors 502-506 associated with a subject, each sensor being operable to obtain one or more biomarkers of the subject, as disclosed above. The system also comprises a processing unit 500 for processing the biomarkers and coupled to the sensors by means of an appropriate input/output unit 510. The processing unit 500 comprises:
a first register 512 containing a first set of digital biomarkers from a subject obtained from one or more sensors associated with the subject;
a first digital neuro signal generator 514 configured to generate from the first set of digital biomarkers a first digital neuro signature for that subject;
a first digital neuro fingerprint generator 516 configured to generate from the first digital neuro signature a first digital neuro fingerprint, the first digital neuro fingerprint being a comparison of the biomarkers in the first digital neuro signature with threshold values associated with the first set of biomarkers;
an identifying processor 518 configured to identify from the first digital neuro fingerprint a first condition of the subject.

The input unit 510 of the processing unit 500 is also configured to receive a second set of biomarkers from one or more sensors 502-506, which may be the same sensors, the same type of sensors or different sensors/sensor types.

The processing unit 500 also includes:
a second digital neuro signature generator 530 configured to generate from a second set of biomarkers a second digital neuro signature;
a second digital neuro fingerprint generator 532 configured to generate from the second digital neuro signature a second digital neuro fingerprint, the second digital neuro fingerprint being a comparison of the biomarkers in the second digital neuro signature with threshold values associated with the second set of biomarkers;
a data pattern identifier 540 configured to identify in the first and second digital neuro fingerprints any data patterns.

The processing unit 500 is configured to utilise any identified patterns in the first and second digital fingerprints to identify the second condition in the first digital neuro fingerprint.

The processing unit 500 may also include a prototypical first digital neuro fingerprint generator 520 configured to generate from a set of first digital neuro fingerprints obtained from a set of subjects a prototypical first digital neuro fingerprint, and a prototypical second digital neuro fingerprint generator 522 configured to generate from a set of second digital neuro fingerprints a prototypical second digital neuro fingerprint. In such an embodiment, the processing unit 500 is configured to identify any data patterns from the first and second prototypical digital neuro fingerprints.

In some embodiments, the processing unit 500 comprises a new first digital neuro fingerprint generator 550 configured to generate a new first digital neuro fingerprint from any data pattern matches determined in the identification step. The new first digital neuro fingerprint is indicative of the second condition in the subject.

As described above, the first and second digital neuro fingerprint generators 516, 532 are preferably configured to form the first and second digital neuro fingerprints as an array of elements, each associated with a biomarker, and in which each element has a value indicative of a deviation of the measured biomarker relative to the threshold, preferably as pixels of a display. For this purpose, the processing unit 500 may comprise an optical pattern identification unit 560.

The processing unit 500 may also include a combined digital neuro fingerprint generator 570 configured to generate a combined digital neuro fingerprint from the first and second digital neuro fingerprints. For this purpose, the processing unit 500 is configured to utilise the combined digital neuro fingerprint in the determination of the first and/or second condition of the subject.

The teachings herein enable the generation of new Digital Neuro Fingerprints on the basis of a preferred algorithm, as described above, or on the basis of combinations of existing and/or new sets of biomarkers in an effort to identify within a group of subjects either indicators of a new disease or illness or new indicators of a previously identified illness or disease, or a combination of both. This can enable the identification of subjects potentially susceptible to a disease or illness from a core set of biomarkers, that had not previously been identified or considered. The identification of such subjects can significantly reduce the number of subjects required for clinical trials and can also help provide earlier diagnosis of disease or illness, potentially long before traditionally considered symptoms exhibit themselves in a patient.

The early diagnosis of cognitive impairment, potentially leading to Alzheimer's Disease can provide early treatment and potentially significantly improved medical outcomes compared to existing methodologies and treatments.

Drugs that might then be prescribed to slow or prevent further deterioration, or to treat symptoms might include a cholinesterase inhibitor (such as donepezil, rivastigmine or galantamine), memantine (optionally in combination with a cholinesterase inhibitor), a monoclonal antibody (such as Aducanumab (Aduhelm), BAN2401, gantenerumab (optionally in combination with solanezumab), solanezumab (optionally in combination with gantenerumab), a sigma-1 receptor agonist (optionally also M2 autoreceptor antagonist or NMDA receptor antagonist, such as ANAVEX2 (blarcamesine), AVP-786 or AXS-05), an SV2A modulator (such as AGB101 (low-dose levetiracetam), a mast-cell stabiliser (such as ALZT-OP1 (cromolyn+ibuprofen)), an anti-inflammatory (such as ALZT-OP1 (cromolyn+ibuprofen)), a RAGE antagonist (such as azeliragon), a glutamate modulator (such as BHV4157 (troriluzole)), a D2 receptor partial agonist (such as brexpiprazole), serotonin-dopamine modulator (such as brexpiprazole), an amyloid vaccine (such as CAD106), a bacterial protease inhibitor (such as COR388), a selective serotonin reuptake inhibitor (such as escitalopram), an antioxidant (such as Ginkgo biloba), a plant extract (such as Ginkgo biloba), an alpha-2 adrenergic agonist (such as guanfacine), an omega-3 fatty acid (such as icosapent ethyl (IPE), which is a purified form of eicosapentaenoic acid), an angiotensin II receptor blocker (such as losartan), a calcium channel blocker (such as amiodipine), a cholesterol agent (such as atorvastatin), a combination of an angiotensin II receptor blocker (such as losartan), a calcium channel blocker (such as amiodipine), a cholesterol agent (such as atorvastatin) with or without exercise, a tyrosine kinase inhibitor (such as masitinib), an insulin sensitiser (such as metformin), a dopamine reuptake inhibitor (such as methylphenidate), an alpha-1 antagonist (such as mirtazapine), an acetylcholinesterase inhibitor (such as octohydro-aminoacridine succinate), a ketone body stimulant (such as tricaprilin), a caprylic triglyceride (such as tricaprilin), a Tau protein aggregation inhibitor (such as AADvac1 or TRx0237 (LMTX)), a positive allosteric modulator of GABA-A receptors (Zolpidem and zoplicone), or BPDO-1603, or combinations of any of these to be administered either together or separately.

Depending on the results, the system may suggest a pharmaceutical intervention, change to an already implemented pharmaceutical intervention (such as change to a dosage or administration regime), and/or may indicate whether or not an intervention continues to be effective.

The system also offers the possibility to a physician to investigate scores obtained in individual areas of the tests in order to determine an optimal intervention for that individual.

The system can thus be used to diagnose an individual with mild cognitive impairment or AD or to predict whether an individual with mild cognitive impairment will convert to AD in due course. It can also be used to assist a physician with prescribing appropriate interventions and/or help to determine whether an already prescribed intervention is working. The system may therefore assist a physician by suggesting starting an intervention, stopping an intervention, or changing an intervention, pharmaceutical or otherwise. It may suggest an appropriate frequency and/or dose of a pharmaceutical intervention or specific drug to be administered to the individual and/or may suggest an appropriate route of administration of a pharmaceutical intervention for that individual. This applies to the specific pharmaceutical interventions mentioned above, for example in Example 4, and to all other potential pharmaceuticals whether or not disclosed herein.

One of the significant advantages of the system described herein is that it is able to assess cognitive capabilities in a single test as compared to the standard neurophysiological assessments currently used in diagnosing AD. As a result, cognitive function measurements can be administered in approximately 10 minutes as compared to 2 hours for the traditional neurophysiological assessments (e.g., MMSE, ADAS-Cog).

The disclosures in U.S. patent application No. 63/277,456, from which this application claims priority, and in the abstract accompanying this application are incorporated herein by reference.

1. A method of determining the condition of a person, comprising:
obtaining a first set of digital biomarkers from one or more sensors associated with a subject,
generating from the first set of digital biomarkers a first digital neuro fingerprint, the first digital neuro fingerprint being a comparison of the first set of biomarkers with threshold values associated with the first set of biomarkers;
identifying from the first digital neuro fingerprint a first condition of the subject;
obtaining a second set of biomarkers from one or more sensors and generating therefrom a second digital neuro fingerprint, the second digital neuro fingerprint being a comparison of the second set of biomarkers with threshold values associated with the second set of biomarkers;
identifying from the second digital neuro fingerprint a second condition of the subject, different than the first condition of the subject;
identifying in the first and second digital neuro fingerprints any data patterns; and
using the identified data patterns in the first and second digital fingerprints to identify indicators of the second condition in the first digital neuro fingerprint.

2.-6. (canceled)

7. A method according to claim 1, wherein any the identified data patterns in the first and second digital fingerprints are used to enhance a determination of the first condition in the first digital neuro fingerprint.

8. A method according to claim 1, wherein the threshold values are representative of an average value of that biomarker in a group of subjects considered to have a predetermined state.

9. A method according to claim 8, wherein the predetermined state is a healthy state.

10. (canceled)

11. A method according to claim 1, including the steps of:
generating a new first digital neuro fingerprint from any data pattern matches determined in the identification step, wherein the new first digital neuro fingerprint is indicative of the second condition in the subject.

12. A method according to claim 1, wherein the digital neuro fingerprints are formed of a plurality of elements, each associated with a biomarker, and in which each element has a value indicative of a deviation of the measured biomarker relative to the threshold.

13. A method according to claim 1, wherein the values of each of the digital neuro fingerprints are in a given range.

14. A method according to claim 13, wherein the value of each element is an optical value.

15. A method according to claim 14, wherein the optical value is a color that changes in dependence on the deviation of a measured biomarker relative to the associated threshold.

16. A method according to claim 15, including the step of identifying patterns in the first and second digital neuro fingerprints by optical pattern recognition.

17. A method according to claim 1, wherein the first and second sets of biomarkers are obtained from different sensors or sensor sets.

18. A method according to claim 1, including the step of generating a combined digital neuro fingerprint from the first and second digital neuro fingerprints, and using the combined digital neuro fingerprint in the determination of the first and/or second condition of the subject.

19. A method according to claim 1, including the step of generating a new digital neuro fingerprint from the first and second digital neuro fingerprints, and using the new digital neuro fingerprint in the determination of the first and/or second condition of the subject.

20. A method according to claim 1, wherein the first and/or second set of biomarkers are obtained from one or more of: a smartphone, a tablet computer, a smart watch, a smart bracelet, a pair of smart glasses, and a camera.

21. (canceled)

22. (canceled)

23. A method according to claim 1, wherein the digital neuro fingerprints are formed of a plurality of elements, each associated with a biomarker, and in which each element has a value indicative of a deviation of the measured biomarker relative to the threshold; and wherein a match is identified if a number of elements in the first and second digital neuro fingerprints exceeds a set proportion.

24. A method according to claim 1, wherein a match is identified if a number of elements in the first and second digital neuro fingerprints exceeds 80% of the total number of elements in at least one of the digital neuro fingerprints.

25. A method according to claim 1, wherein the step of identifying a pattern generates and uses Shapley Values.

26. A system for determining the condition of a person comprising:
one or more sensors associated with a subject, the or each sensor being operable to obtain one or more biomarkers of the subject;
a processing unit for processing the biomarkers, the processing unit comprising:
a first register containing a first set of digital biomarkers from a subject obtained from one or more sensors associated with the subject;
a first digital neuro fingerprint generator configured to generate a first digital neuro fingerprint, the first digital neuro fingerprint being a comparison of the biomarkers with threshold values associated with the first set of biomarkers;
an identifying unit configured to identify from the first digital neuro fingerprint a first condition of the subject;
an input configured to receive a second set of biomarkers from one or more sensors;
a second digital neuro fingerprint generator configured to generate a second digital neuro fingerprint, the second digital neuro fingerprint being a comparison of the biomarkers with threshold values associated with the second set of biomarkers;
a data pattern identifier configured to identify in the first and second digital neuro fingerprints any data patterns;
the processing unit being configured to use any identified patterns in the first and second digital fingerprints to identify the second condition in the first digital neuro fingerprint.

27.-75. (canceled)
